(19) United States
(12) Patent Application Publication
(10) Pub. No.: US 2012/0150188 A1
(43) Pub. Date: Jun. 14, 2012

(54) ABSORBABLE BONE ADHESIVE APPLICATOR

(76) Inventor: Stephen McCarthy, Tyngsboro, MA (US)

(21) Appl. No.: 13/263,409

(22) PCT Filed: Apr. 16, 2010
(86) PCT No.: PCT/US10/31347

§ 371 Date: Feb. 29, 2012

Related U.S. Application Data
(60) Provisional application No. 61/170,327, filed on Apr. 17, 2009.

(51) Int. Cl. A61B 17/80; A61B 17/88

(52) U.S. Cl. 606/70; 606/281

(57) ABSTRACT

The instant invention provides of a novel method for repairing bone defects using a polymer blend as well as apparatus for performing such a method. The method includes dispensing a polymer blend either directly on the bone tissue as the fixation means; or secondly in combination with a bone plate, the polymer acting as an adhesive

This application claims priority from U.S. Provisional Patent Application Ser. No. 61/170,327, filed on Apr. 17, 2009, entitled Absorbable Bone Adhesive Applicator, whose disclosure is hereby incorporated by reference in its entirety.

### BACKGROUND

Surgical repairs of bone tissue and reattachment and closure of bone in cranial surgeries requires approximation and fixation and of bone tissue most often by means of mechanical fastening systems such as screws and plates. Often the surgery presents particular challenges to the practitioner due to tissue conditions, limited space, and access of the surgical site. To overcome the challenges presented in surgery many of surgical methods, tools and materials have been developed to overcome these issues. In addition many of the bones are presented in a manner that makes it difficult or prevents mechanical fixation due to a lack of tissue surface area or tissue substrate depth to afford a adequate secure location for the device.

In addition, standard mechanical attachment in the context of bone repairs includes a series of labor intensive and sometimes complicated tasks that are required to complete the repair. Although some of the mechanical products offered today have helped to simplify this procedure, the mechanical attachments are still often time consuming, bulky and cumbersome.

Furthermore, the type, weight and amount of material used in plating are other sources of concern. It is preferred by the surgical community to use materials that compatible with tissue and be absorbable. Many of the existing mechanical systems are metal and non-resorbable. The material used in the instant invention and the associated methods and devices overcome may of the shortcomings of mechanical fixation of bone described above.

What is needed therefore is a method and apparatus for dispensing a biocompatible lightweight plastic material that is absorbable. The amount of material can be reduced by not having overlapping redundant structures for fixing the bone. Such a method and apparatus would eliminate many of the surgical steps required to place mechanical fixation devices. In particular drilling, tapping of the tissue substrate and threading or placement of fixation pin or screw would no longer be necessary.

### SUMMARY OF THE INVENTION

The instant invention provides of a novel method for repairing bone defects using a polymer blend as well as apparatus for performing such a method. The method includes dispensing a polymer blend either directly on the bone tissue as the fixation means; or secondly in combination with a bone plate, the polymer acting as an adhesive. The polymer blend can include any number of polymer blends but is preferably the polymer bend(s) described in co-pending patent application U.S. patent application Ser. No. 11/787,076 entitled: Novel Biodegradable Bone Plates And Bonding Systems, which is hereby incorporated by reference in its entirety. In order to heat and dispense the adhesive material a device is required. The device is in the form a hand held surgical tool that can process the heating of the adhesive material and then deliver the material to surgical site. The device used for delivery of the materials includes novel control mechanism and features to implement an effective bone repair.

In one aspect, the invention contemplates a device for providing a surgical adhesive for repairing bone plates having an elongate shaft with a proximal and a distal end, an ergonomic handle at the proximal end of the shaft for actuating the delivery, and dispensing of the flowable adhesive material. The device further includes a customizable dispensing tip at the distal end of the shaft, for customizing the material configuration to match the desired results for making the surgical repair, a control mechanism for controlling flow of the surgical adhesive and a user interface to said control mechanism. In one embodiment the control mechanism controls flow rate.

In another aspect, the invention constitutes a method for securing a bone plate to bone. The method comprising applying an absorbable adhesive polymer material in a in flow able state to the bone, positioning the bone plate and holding the bone plate in place while the adhesive hardens.

In a further aspect the invention contemplates a method of securing two or more fragments of bone together by applying an absorbable adhesive polymer to a first bone fragment, contacting a second bone fragment to said first bone fragment and holding said bone fragments together until the absorbable adhesive polymer hardens.

### BRIEF DESCRIPTION OF THE DRAWINGS

FIG. 1 depicts a conventional bone plate fixation by securing the supporting plate using screws.

FIG. 2 depicts a bone plate fixation using a series of support plates and an adhesive such as biodegradable melt adhesives.

FIG. 3 depicts a plate-less bone fixation using only an adhesive such as biodegradable melt adhesives.

FIG. 4 is a comparison of biomechanical data of healed osteotomies using conventional fixation, adhesive only and bone plate with adhesive techniques.

FIG. 5 shows a histological analysis of osteotomies treated using various securing systems at 6 weeks post surgery

FIG. 6 shows a histological analysis of osteotomies treated using various securing systems at 3 months post surgery

FIG. 7 shows the average operative time using various techniques.

FIG. 8 is a schematic drawing of one embodiment of the adhesive applicator of the invention.

FIG. 9. is a control diagram of the control system of the adhesive applicator of the invention.

### DETAILED DESCRIPTION OF THE INVENTION

### Method of Application and Repair

The method applying the absorbable adhesive polymer material for the purpose of repairing bone and the associated applicator device differ from existing surgical methods and apparatus. Firstly the mechanical plating systems utilized screws, pins or some fastening device to affix the plate to the bone and involves a secondary operation of drilling holes to receive fastening device into the bone. In the subject invention no bone drilling is required. The plate is affixed using the adhesive in flow able state and is held in place while it hardens. In another embodiment of the invention the adhesive alone can be applied to the bone without a plate and the adhesive alone is used to affix the bone together or fill in a void. The method joins and affixes the bone segments by placing the adhesive material along each of adjoining bone surfaces. It can be utilized in a number of geometrically varied bone shapes.

This method can be used in conjunction with various medical procedures. This includes, for example, osteotomies of the frontal bone and infraorbital rims on the skull. In these type of surgeries bone segments are surgically removed and replaced (craniotomy-like procedure) with the absorbable adhesive material. Applicants have found that this procedure results in operating time reduction. and remarkable improvements in tissue healing based on histological review of the tissue.

### Adhesive Applicator Device:

The need to have a device that meets the need of the surgical method of fixing the bones with plates or without requires overall it have ergonomic user controls, and be sterilizable. The invention disclosed is a tool that delivers a suture that is more predictable and automated one step process like surgical stapling devices. The delivery mechanism is comprised of an elongate shaft that has an ergonomic handle at one end for actuating the delivery, and dispensing of the flowable adhesive material. For optimized user control it is desirable to have various endofector dispensing tips to customize the material configuration to match the desired results for making the surgical repair. A drawing of the device shown here describes some of these features.

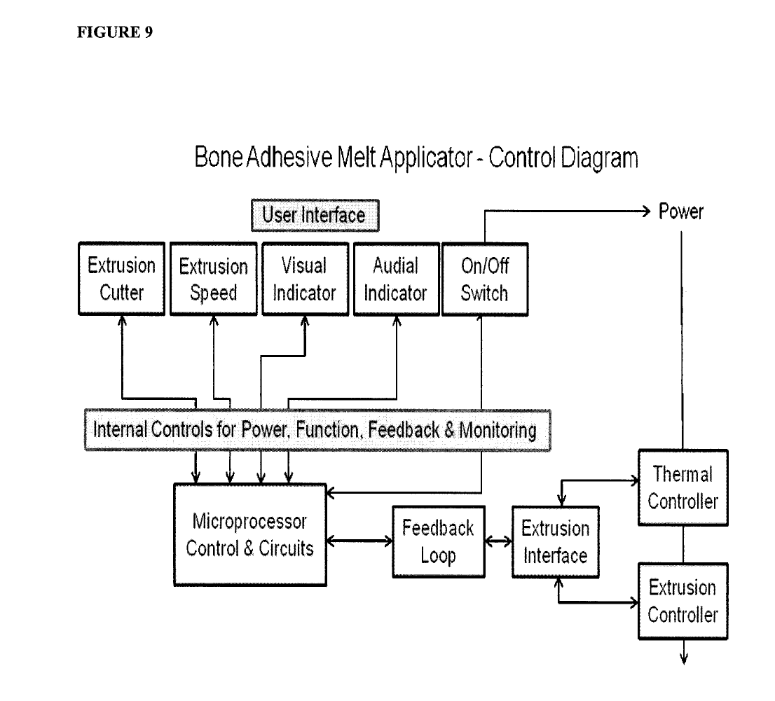

Other surgical controls include material flow rate, cutting off of material, and a start and stop of material flow. The adhesive applicator includes user interface for these controls. Other controls of the device are automated by means of an electric circuit with feedback and control loops to achieve desired results. Included in the electric controls are user feedback indicators both audible and visual. Other internal controls are preset with no user interface. FIG. 9 shows an example of a control schematic of the adhesive applicator.

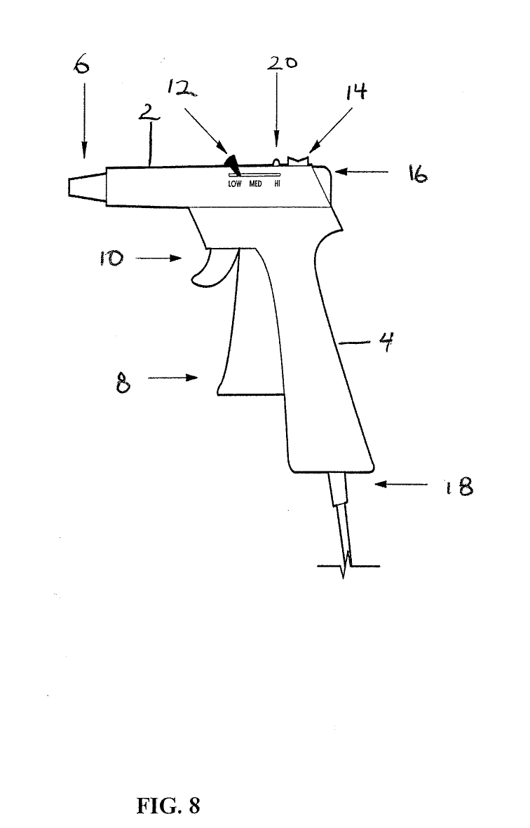

FIG. 8 shows a schematic of one embodiment of the device. The device includes means for heating, mixing and advancing the material. The material is inserted into the device at proximal portion (toward the user) and is advanced to the distal end of (working end) the device. The mechanism for dispensing is a spiral/screw mechanism that delivers adhesive pellets from a feed unit such as a hopper into the body of the handle and through to the distal tip of the device. The material is heated to a flowable temperature. The glass transition temperatures of the polymers and their blends can be estimated using the following formula”.

More specifically, as shown in FIG. 8 the device comprises an elongate shaft 2 with a proximal and a distal end, an ergonomic handle 4 at the proximal end of the shaft for actuating the delivery, and dispensing of the flowable adhesive material. The device further includes a customizable dispensing tip 6 at the distal end of the shaft, for customizing the material configuration to match the desired results for making the surgical repair. The dispensing tip is preferably made out of insulated and/or heat resistant material. The device further comprises a control mechanism for controlling flow of the surgical adhesive. In the illustrated embodiment the control mechanism is comprised of trigger 8 that activates the flow of the material and a cut off trigger that 10 that immediately stops the flow of material from the device. Additionally, a material flow speed control 12 is included to better control the rate of low of the material, At the proximal end of the shaft of the illustrated embodiment, the device includes a material feed 16. for feeding material into the applicator. In a preferred embodiment the flowable material exists in a solid or semi-solid state at room temperature, is fed into the device via material feed 16, is heated via a heating element located within the shaft 2 of the adhesive applicator and is dispensed at the tip 6. user interface to said control mechanism. Power is supplied to the system via an electrical power supply 18, which is controlled by a switch 14. The device can optionally include a power indicator lamp 20, for indicating to the user when the power is on.

Equations to identify the desired glass transition of the blends:

$$\frac{1}{{T}_{g}} = \frac{{M}_{1}}{{T}_{g 1}} + \frac{{M}_{2}}{{T}_{g 2} }$$

Where T_{g} is the desired glass transition (T_{g}) of the blend
M₁ is the mass of material 1
M₂ is the mass of material 2
T_{g1} is the glass transition temperature of material 1
T_{g2} is the glass transition temperature of material 2

Furthermore the blend can be optimized to achieve a specific glass transition temperature as shown in Table 1 below:

TABLE 1
Example of blending ratio to achieve specific T_{g} for a blend

| Desired T_{g} of | Ratio of PCL | Ratio of PLA |
|---|---|---|
| Blend (° C.) | (T_{g}~−60° C.) | (T_{g}~70° C.) |
| 70 | 0.0 | 1 |
| 60 | 0.077 | 0.923 |
| 50 | 0.1846 | 0.8154 |
| 40 | 0.3462 | 0.6538 |
| 30 | 0.6154 | 0.3846 |

Furthermore, the output of the device can be modulated using a standard single screw output equation as follows:

Standard single screw output equations:

Q=Q_{drag}−Qₚᵣₑₛₛᵤᵣₑ

$${Q}_{d} = \frac{{F}_{d} {π }^{2} {D}^{2} N h ( 1 - \frac{n e}{t} ) sin ϑcos ϑ}{2}$$

$${Q}_{p} = \frac{{F}_{p} π D {h}^{3} ( 1 - \frac{n {e}^{2}}{t} ) {sin}^{2} ϑ}{12 µL}$$

### Where:

Q_{d}=drag flow (pumping term)
Qₚ=pressure flow (resisting pumping)
F_{d}=0.140 (h/w)²−0.645 (h/w)+1
Fₚ=0.162 (h/w)²−0.742 (h/w)+1
D=screw diameter
N=Screw speed
h=screw's meter section channel depth
w=screw channel width
n=number of flights on the screw
e=thickness of flights
θ=flight helix angle
μ=viscosity of melt
L=length of metering section being investigate

For a fixed speed of 50 rpm and a standard screw the output can also be calculated as Out put Q=0.277 (screw diameter)³ (kg/hr).

It should be understood that the procedures and mechanism of the invention could include other methods and delivery devices for providing a bone adhesive.

### Examples

### Materials

Materials used for the study were Polylactic acid (Nature Works 5040D, Cargil), Polycaprolactone (440744, Aldrich), Delta System™ (Stryker).

### Sample Preparation

### Melt Blending:

Blends of PLA with PCL with the ratio 80:20 were prepared in melt blender (HAKKE Rheocord, TYP-557-0029, capacity 60 grams by weight). The resin was dried for 6 hours at 60° C. in the vacuum oven. The blending temperature was selected as the melt temperature for the resins (175° C.). The rpm was set at 40±0.5. The cycle time was set as 3 minutes.

### Compression Molding:

Plate with adhesive plates were prepared from Polylactic acid (PLA) blended with Polycaprolactone (PCL). The dried (40° C. for 24 hours) pellets were compression molded in an Aluminum mold. The compression molding plates and the mold were preheated for 5 min at 175° C. Before applying the entire pressure the mold was vented to remove any trapped air or gases between the pellets. A compression pressure of 2 MPa was applied for 3 minutes. This step was followed by removal of mold plates and cooling them in air at room temperature of approximately 23° C. The compression molded plates were then cut into strips of 7.5 mm×68 mm for tensile testing of the adhesive's strength.

### Animal Model:

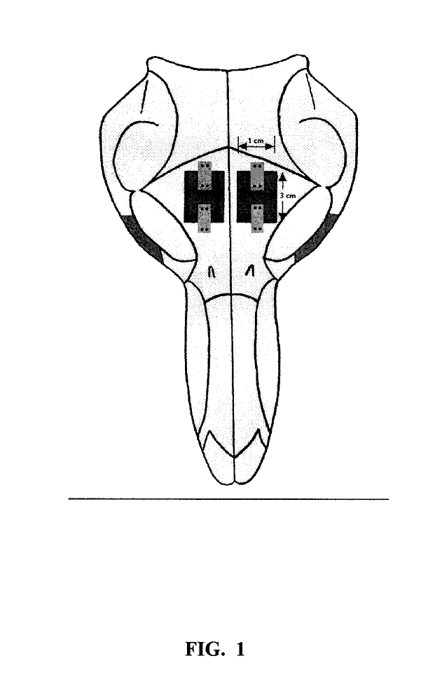

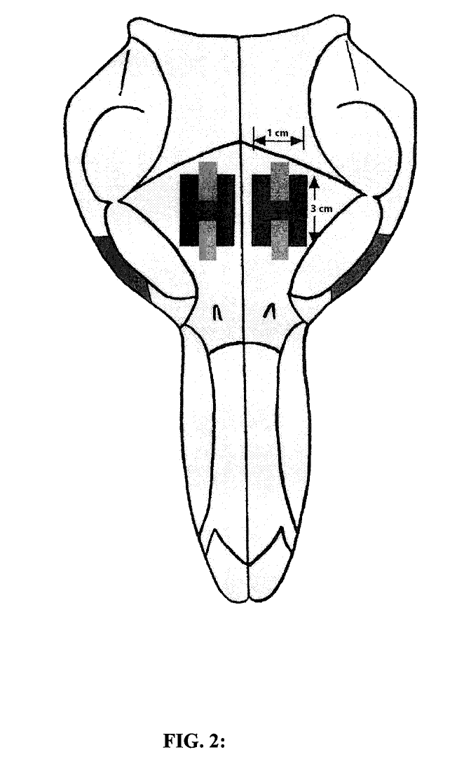

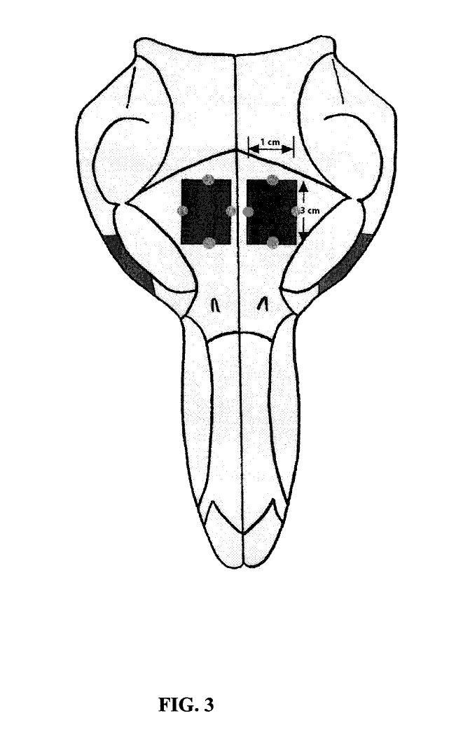

Skeletally-immature goat models (6 months of age) underwent osteotomy of the frontal bone and infraorbital rims. The goats resumed their normal activities following surgery for 6 weeks, 3 months, and 6 months prior to sacrifice. The goats were then euthanized and the healed osteotomies were carefully removed and analyzed. FIG. 1, FIG. 2 and FIG. 3 are the graphical representation of the methods used for fixation of the osteotomies. The larger (3 cm×1 cm) rectangles are representative of the bone fragments to be re-attached to the underlying bone. Specifically FIG. 1 shows a standard procedure where a bone fragment is attached using a combination of plates and screws. FIG. 2 shows a modified procedure where the bone is attached with the assistance of plates, the plates having been attached to the bone using adhesive dispensed using the devices and methods of the invention. FIG. 3 shows the attachment of the bone fragment to the underlying bone using a further modified procedure that avoids the use of bone plates.

The smaller rectangles in FIGS. 1 and 2 are representative of bone plates. The small dots in FIG. 1 depict the use of screws to secure the bone plates to the bone fragment as well as the underlying bone. The circular areas in FIG. 3 depict areas where adhesive has been deposited.

### Tensile Testing

Tensile testing was performed according to modified ASTM D 882. The grip separation rate was kept constant at 2.0 inches/min. All testing was performed by using Tensile Testing machine, model number 6025 and a load cell of 50 kN. The machine was operated in tension mode. The lead time for testing was about 3-5 days. The fresh bone samples were transported and stored in subzero temperatures for uniformity.

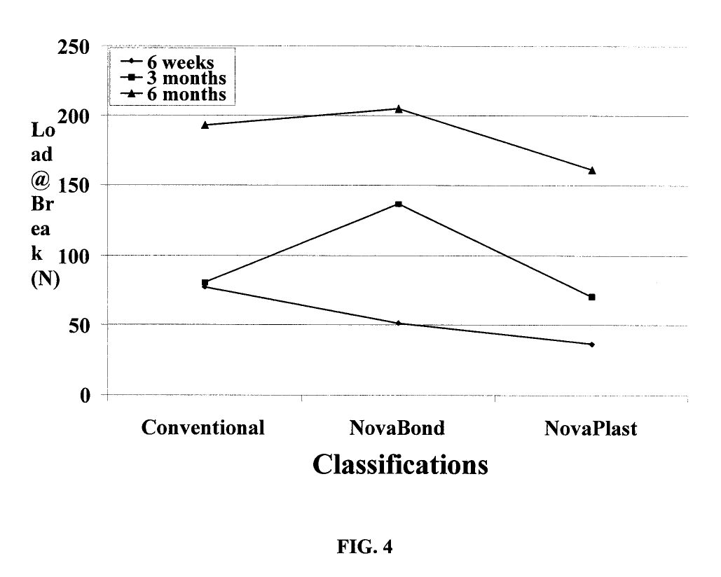

FIG. 4 is the graphical representation of the data received through biomechanical analysis of the healed osteotomies. The adhesive used for the “adhesive only” and “plate with adhesive” systems was a Polycaprolactone melt. In all three bone fixation systems a general trend of increase in bone strength over the period of time was observed. The conventional system demonstrated similar bone strength values from 6 weeks to 3 months of healing. It was hypothesized that from 6 weeks to 3 months there is no significant degradation of the screw and implanted supporting plate. It is assumed that the bony tissues grow around the screw hole during the healing, leaving a tiny cavity at the location of the screw. By 6 weeks the actual osteotomy is fixed making the screw hole the weakest link. A similar explanation can be provided for the 3 month data with the conventional system. In addition, the initial higher strength of the conventional system for the first 6 weeks as observed in FIG. 4 could be the result of the strength of the securing plate added to the healing osteotomy. In the case of the Adhesive only and Plate with adhesive systems, there is no requirement for drilling holes (required for the screw in the conventional system) as the biodegradable melt adhesive is applied directly to the bony surface.

FIG. 4 clearly depicts a continuous improvement at the osteotomy site over the period of time. In the case of Adhesive only (a/k/a Novabond) and Plate with adhesive (a/k/a Novaplast), the increase in load-bearing capacity over 6 weeks, 3 months, and 6 months periods is a direct reflection of bone growth and healing of the osteotomy sites. In three months; time Plate with adhesive offers comparable results to the conventional system. However, the Adhesive only system demonstrates twice the strength of bone compared to the conventional and Plate with adhesive systems within the same time frame of 3 months. It was hypothesized that with the conventional and Plate with adhesive systems, the presence of supporting implant (plate) restricts bone growth. The surface area occupied by the supporting plate is not available for tissues growth. It is hypothesized that further growth of the bony segments will be now depending on the degradation of the implant. The hypothesis was strengthened by almost comparable results at the 6 month period, depicted in FIG. 4. During the 6 month time frame most of the polymer matrix (plates/adhesive/screws) appears to have degraded, allowing the entire area to be available for bony growth. It can be argued that by 6 months and beyond, the healing process relies on the degradation and resorption of the individual polymer and is independent of the specific system adapted to secure the osteotomies.

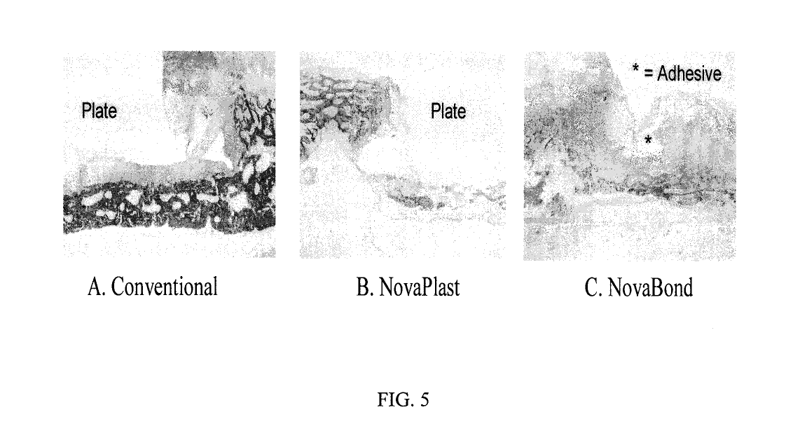

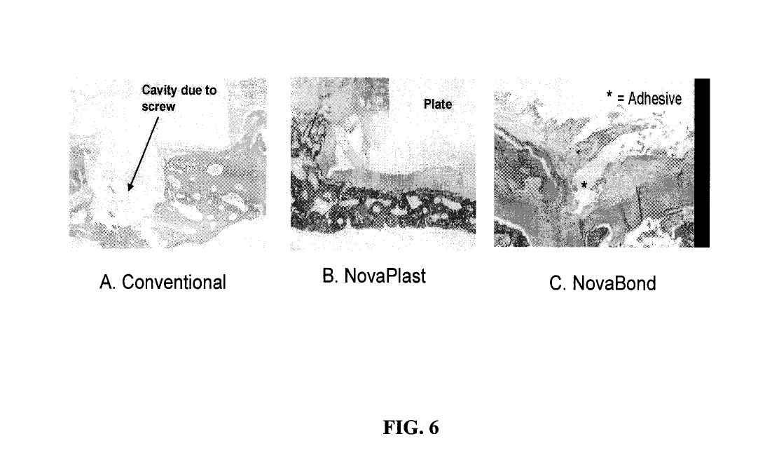

To better understand the results at the selected time frames, histological assessment of the bone segments were performed. In FIG. 5 all three systems at 6 weeks can be observed. The dark solid region indicates the presence of bone. At six weeks the Conventional system and Plate with adhesive appear to be similar. Adhesive only demonstrated a reduced amount of polymer at the osteotomy site. At 3 months time (FIG. 6), image A shows the lack of growth of bone at the screw sites resulting in a weaker section. Image B demonstrates the application of the Plate with adhesive plate and adhesive without the need for screw fixation. In both the Conventional and Plate with adhesive systems there is no bone growth observed in the area occupied by the supporting plate/implant. However, in image C, in which only the adhesive is used for fixation, the growth of fresh bone over the adhesive is seen. This image proves the hypothesis of greater bone growth with the Adhesive only system resulting in greater load bearing capacity at 3 months.

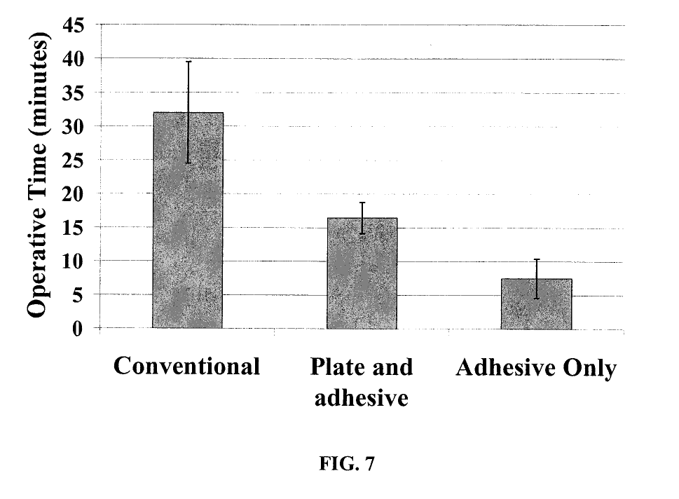

The FIG. 7 graphically compares the typical operative time for the three fixation systems under investigation. Four goats for each system were operated and the operative time was noted. It was observed that the conventional system required approximately 32±8 minutes, Plate with adhesive required 16±2 minutes, and Adhesive only required 7±3 minutes. The higher time requirement with the conventional system is attributed to the positioning of the plate, drilling of holes, and application of the screws. In the event of repositioning of the plates the entire process has to be repeated, thus involving more time. In the case of Plate with adhesive, the use of adhesive rather than screws facilitates the procedure such that it can be performed in half the time as the conventional system. With the Adhesive only system, all the support is provided by the adhesive; thus no positioning of the plates or drilling of holes for screws is required making it four times faster than the conventional system. Significantly decreased operative time required with Adhesive only makes it more desirable for the surgeons as well as the patients.

Application and comparison of different fixation systems to secure an osteotomy site were successfully performed and analyzed. The use of screws and plates in the Conventional system provides 33% greater strength than Adhesive only and 53% greater strength than Plate with adhesive for the initial six weeks. However, availability of more surface area for bone growth with the Adhesive only system at 3 months provides 158% greater strength compared to the conventional system. Histological images show the formation of new bone growth around and over the adhesive bond. The presence of plates restricts the normal growth of the bone until the supporting plate degrades. At the six month time period it can be concluded that healing/growth of the bone becomes independent of any specific securing system and remains dependent on individual polymers and their formulations. Furthermore, the fixation operative time requirement is 50% less in the Plate with adhesive system and 75% less in Adhesive only system when compared to the conventional screw and plate fixation system.

1. A device for providing a surgical adhesive for repairing bone plates comprising:
(a) an elongate shaft having a proximal and a distal end;
(b) an ergonomic handle at the proximal end of the shaft for actuating the delivery, and dispensing of the flowable adhesive material.
(c) a customizable dispensing tip at the distal end of the shaft, for customizing the material configuration to match the desired results for making the surgical repair.
(d) a control mechanism for controlling flow of the surgical adhesive;
(e) a user interface to said control mechanism.

2. The device of claim 1 wherein the control mechanism controls flow rate;

3. A method for securing a bone plate to bone comprising:
(a) applying an absorbable adhesive polymer material in a in flow able state to the bone;
(b) positioning the bone plate
(c) holding the bone plate in place while the adhesive hardens.

4. A method of securing two or more fragments of bone together comprising:
(a) applying an absorbable adhesive polymer to a first bone fragment;
(b) contacting a second bone fragment to said first bone fragment;
(c) holding said bone fragments together until the absorbable adhesive polymer hardens.
